(19) United States
(12) Patent Application Publication
(10) Pub. No.: US 2026/0315016 A1
(43) Pub. Date: Oct. 15, 2026

(54) MOBILE EXAMINATION APPARATUS

(71) Applicant: CANON KABUSHIKI KAISHA, Tokyo (JP)

(72) Inventors: YUKI NIWAMAE, Kanagawa (JP); TAKAHIRO YAMAMOTO, Kanagawa (JP); TAKAYUKI SHIBATA, Kanagawa (JP); SATOSHI SHINATA, Kanagawa (JP); SAKIKO TANIMOTO, Shizuoka (JP); SEISHIRO TAKANO, Kanagawa (JP); TETSUYA SEKINE, Kanagawa (JP); EMIKO KAMIYAMA, Tokyo (JP); SHOSAKU KAWASHIMA, Kanagawa (JP); HAZUKI TOKI, Tokyo (JP)

(21) Appl. No.: 19/467,052

(22) Filed: Feb. 2, 2026

Related U.S. Application Data
(63) Continuation of application No. PCT/JP2024/031194, filed on Aug. 30, 2024.

(30) Foreign Application Priority Data
Sep. 5, 2023 (JP) 2023-143452

(51) Int. Cl. A61B 6/00; A61B 8/00

(52) U.S. Cl. CPC A61B 6/4405; A61B 6/4429; A61B 8/4405; A61B 8/4416

(57) ABSTRACT

According to an aspect of the present disclosure, a mobile examination apparatus includes a housing, a column coupled to the housing and configured to extend and retract in a vertical direction, a first arm coupled to the column and configured to extend and retract in a longitudinal direction, a radiation emitting unit supported by the first arm and configured to emit radiation, and a mobile unit configured to support the housing and move on a floor surface, wherein the first arm is folded relative to the column while supporting the radiation emitting unit, and the housing houses the radiation emitting unit, the first arm, and the column therein.

### CROSS-REFERENCE TO RELATED APPLICATIONS

This application is a Continuation of International Patent Application No. PCT/JP2024/031194, filed Aug. 30, 2024, which claims the benefit of Japanese Patent Application No. 2023-143452, filed Sep. 5, 2023, both of which are hereby incorporated by reference herein in their entirety.

### BACKGROUND

### Field of the Technology

The present disclosure relates to a mobile examination apparatus that is intended for purposes, such as medical diagnosis, and capable of being moved.

### Description of the Related Art

In recent years, mobile examination apparatuses with excellent mobility have been developed, and it has become possible to move the apparatus to a subject’s home and perform radiographic examinations or the like for home medical care.

For example, Japanese Patent Publication No. 2014‑138645 describes an X‑ray imaging apparatus in which a housing unit capable of housing an X‑ray source is provided with wheels, a column unit, and a grip portion to enhance portability. When a user moves the X‑ray imaging apparatus, the X‑ray source is housed in the housing unit, and the user holds the grip portion and tows the apparatus.

In the case of the apparatus described in Japanese Patent Publication No. 2014‑138645, when the user performs an X‑ray examination after transportation of the apparatus, it is necessary to attach the X‑ray source, which has been housed in the housing unit, in the vicinity of a distal end of the grip portion. Further, during the transportation, the X‑ray source is required to be detached from the vicinity of the distal end of the grip portion and housed in the housing unit.

### SUMMARY

Issues intended to be addressed by an embodiment disclosed in the specification and drawings are not limited to those described above. Issues corresponding to effects of respective configurations described in the embodiment described below may also be regarded as other issues.

The present disclosure is directed to providing a mobile examination apparatus that facilitates installation.

According to an aspect of the present disclosure, a mobile examination apparatus includes a housing, a column coupled to the housing and configured to extend and retract in a vertical direction, a first arm coupled to the column and configured to extend and retract in a longitudinal direction, a radiation emitting unit supported by the first arm and configured to emit radiation, and a mobile unit configured to support the housing and move on a floor surface, wherein the first arm is folded relative to the column while supporting the radiation emitting unit, and the housing houses the radiation emitting unit, the first arm, and the column therein.

Features of the present disclosure will become apparent from the following description of embodiments with reference to the attached drawings.

### BRIEF DESCRIPTION OF THE DRAWINGS

FIG. 1A is a perspective view illustrating a configuration of a mobile examination apparatus according to the present disclosure.

FIG. 1B is a perspective view illustrating a configuration of the mobile examination apparatus according to the present disclosure.

FIG. 2 is a diagram illustrating a state of the mobile examination apparatus during transportation.

FIG. 3 is a diagram illustrating a state with a leg unit of the mobile examination apparatus pivoted.

FIG. 4 is a diagram illustrating a state with a column of the mobile examination apparatus moved upward.

FIG. 5 is a diagram illustrating a state with a radiation detector of the mobile examination apparatus taken out.

FIG. 6 is a diagram illustrating a state with a panel unit of the mobile examination apparatus taken out.

FIG. 7 is a diagram illustrating an internal configuration of a main body of the mobile examination apparatus.

FIG. 8 is a diagram illustrating an example during a radiographic examination of the mobile examination apparatus.

FIG. 9 is a diagram illustrating an example during an ultrasound examination of the mobile examination apparatus.

FIG. 10 is a flowchart illustrating an operation of the mobile examination apparatus.

### DESCRIPTION OF THE EMBODIMENTS

An embodiment of the present disclosure will be described below with reference to the drawings.

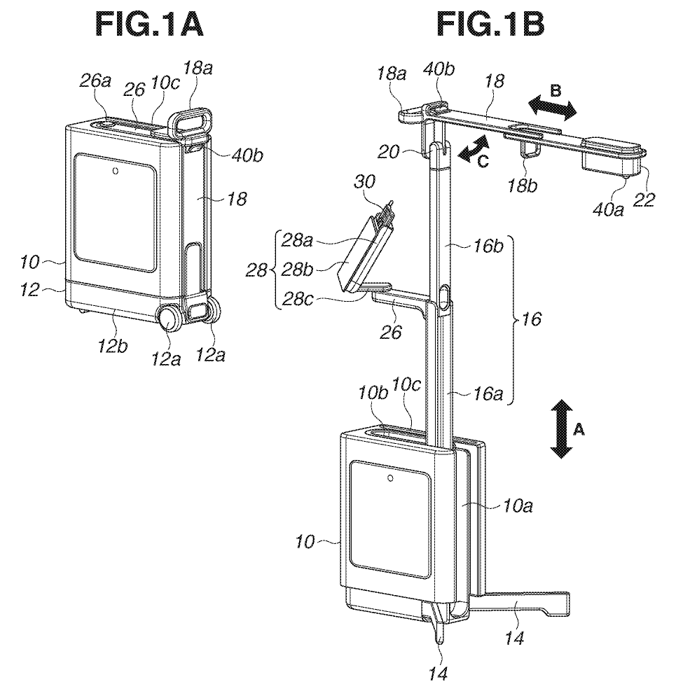

FIGS. 1A and 1B are diagrams illustrating a configuration of a mobile examination apparatus. FIG. 1A is a perspective view of the mobile examination apparatus during transportation. FIG. 1B is a perspective view of the mobile examination apparatus during a radiographic examination.

The mobile examination apparatus according to the present disclosure has a function that is capable of performing a radiographic examination and an ultrasound examination.

The mobile examination apparatus includes a housing 10 having a substantially cuboid shape, a mobile unit 12 detachable from the housing 10 and provided with wheels 12a, a column 16 that is vertically movable and extendable, a main arm 18 that is pivotably coupled to the column 16, and a sub arm 26 capable of supporting a panel unit 28 having a display unit.

During transportation, when neither a radiographic examination nor an ultrasound examination is performed, components, such as the column 16, the main arm 18, and the sub arm 26, are housed in the housing 10 as illustrated in FIG. 1A. The housing 10 houses at least part of each of the components, such as the column 16, the main arm 18, and the sub arm 26. The components, such as the column 16, the main arm 18, and the sub arm 26, may form an upper surface or a side surface of the housing 10.

During a radiographic examination, as illustrated in FIG. 1B, the components, such as the column 16, the main arm 18, and the sub arm 26, are drawn out from the housing 10 and unfolded.

During a radiographic examination, the main arm 18 is disposed at a position higher than the sub arm 26. The main arm 18 and the sub arm 26 are disposed substantially parallel to a floor surface.

The main arm 18, which is pivotably coupled to the column 16, has a radiation emitting unit 22 thereon.

The main arm 18 and the sub arm 26 are disposed in mutually different directions with respect to the column 16. Specifically, the main arm 18 and the sub arm 26 are disposed in opposite directions. More specifically, as illustrated in FIG. 1B, with respect to the plane of the drawing, the main arm 18 is disposed to the right of the column 16, and the sub arm 26 is disposed to the left.

The housing 10 has a function of housing components. During transportation, the housing 10 can house, for example, the radiation emitting unit 22, the panel unit 28, and a radiation detector 24 and an ultrasound probe 30 which are described below. The housing 10 is also capable of housing a power supply, a control apparatus, and other equipment of the mobile examination apparatus.

The housing 10 includes a main‑arm housing unit 10a on the side surface, and a sub‑arm housing unit 10b on the upper surface. The housing 10 may further include a radiation detector housing unit 10c on the upper surface. The main‑arm housing unit 10a may be referred to as a first housing unit. The sub‑arm housing unit 10b may be referred to as a third housing unit. The radiation detector housing unit 10c may be referred to as a fourth housing unit.

The main‑arm housing unit 10a (first housing unit) has a space for housing the radiation emitting unit 22 coupled to the main arm 18 and the column 16 in addition to the main arm 18. Specifically, a recessed portion is formed in the side surface of the housing 10 as the main‑arm housing unit 10a. The main‑arm housing unit 10a has a function of housing the main arm 18, the radiation emitting unit 22, and the column 16 in the recessed portion formed in the side surface of the housing 10. Alternatively, the housing 10 may have a hollow interior, and the main arm 18, the radiation emitting unit 22, and the column 16 may be housed inside the housing 10.

The sub‑arm housing unit 10b (third housing unit) has a space for housing the sub arm 26. Specifically, a recessed portion is formed in the upper surface of the housing 10 as the sub‑arm housing unit 10b. The sub‑arm housing unit 10b has a function of housing the sub arm 26 in the recessed portion formed in the upper surface of the housing 10.

The radiation detector housing unit 10c (fourth housing unit) has a space for housing the radiation detector 24. Specifically, the housing 10 has a hollow interior and a slit is formed in the upper surface of the housing 10. The radiation detector housing unit 10c has a function of sliding the radiation detector 24 from the upper surface into the housing 10 and housing the radiation detector 24.

According to the present embodiment, the housing 10 includes the radiation detector housing unit 10c (fourth housing unit) for housing the radiation detector 24 that detects radiation emitted from the radiation emitting unit 22.

FIG. 1A is the perspective view of the mobile examination apparatus during transportation. The mobile unit 12 includes the plurality of wheels 12a that rotate while being in contact with a floor surface, and a cover 12b that covers a bottom surface of the housing 10. The plurality of wheels 12a is provided with respect to the cover 12b. The plurality of wheels 12a may be tires or casters, and when a user tows the mobile examination apparatus for transportation, the plurality of wheels 12a rotates in contact with the floor surface to facilitate transportation of the mobile examination apparatus. The mobile unit 12 is detachable from the housing 10. For example, when the mobile examination apparatus is transported, the mobile unit 12 is attached to the housing 10 for towing, and upon arrival at a subject’s home, the mobile unit 12 is detached from the housing 10 before entering indoors, and the housing 10 is carried indoors and placed near the subject. By separating the mobile unit 12 that is soiled from the housing 10 that is clean, the indoor environment can be maintained in a sanitary condition.

A lock mechanism (not illustrated) for integrating the housing 10 with the mobile unit 12 may be provided on the cover 12b of the mobile unit 12. When a lock of the lock mechanism is engaged with the housing 10 mounted on the cover 12b of the mobile unit 12, the mobile unit 12 and the housing 10 are integrated together. When the lock of the lock mechanism is disengaged, the mobile unit 12 can be detached from the housing 10.

The column 16 is disposed to be extendable relative to the housing 10. The column 16 includes a movable portion 16a and an extendable portion 16b. The column 16 is set along the side surface of the housing 10, and the movable portion 16a can slide vertically (in a direction A) relative to the housing 10. The extendable portion 16b can slide vertically (in the direction A) relative to the movable portion 16a for extension and retraction. The user can adjust a height of the radiation emitting unit 22 by sliding the movable portion 16a vertically (in the direction A) and by sliding the extendable portion 16b vertically (in the direction A) to extend or retract the extendable portion 16b. During transportation, a lock mechanism (not illustrated) is provided to lock the housing 10 and the column 16 so that the column 16 housed in the housing 10 cannot move vertically.

The column 16 is set along the side surface of the housing 10, and the movable portion 16a can slide vertically (in the direction A) relative to the housing 10. Further, the extendable portion 16b can slide vertically (in the direction A) relative to the movable portion 16a for extension and retraction.

When the column 16 is housed, the extendable portion 16b is slid downward so that the movable portion 16a and the extendable portion 16b overlap each other. Then, with the extendable portion 16b overlapped, the movable portion 16a can be slid downward. The column 16 retracted by sliding the movable portion 16a and the extendable portion 16b downward is shorter than a height of the housing 10. Because a length of the movable portion 16a and a length of the extendable portion 16b are shorter than the height of the housing 10, the column 16 which has been retracted is shorter than the height of the housing 10. Therefore, the column 16 can be housed in the housing 10.

When a radiographic examination is performed, the extendable portion 16b is slid upward, and the column 16 which has been extended has a length equal to the combined lengths of the movable portion 16a and the extendable portion 16b and thus becomes longer than the height of the housing 10.

Leg units 14 are pivotably coupled to a bottom edge of the housing 10. During transportation, the leg units 14 are folded along the housing 10. Due to the mobile unit 12 attached to the housing 10 in such a manner that the leg units 14 are covered by the cover 12b of the mobile unit 12, the leg units 14 are not pivoted. When installing the mobile examination apparatus near a subject, the cover 12b of the mobile unit 12 is detached and the user pivots the leg units 14 toward a side on which the main arm 18 is set. A pivot axis of each of the leg units 14 is substantially parallel to a longitudinal direction of the column 16. The user can pivot the leg units 14 while keeping the leg units 14 in contact with the floor surface, and adjust respective pivot angles of the leg units 14 as desired.

While, in this embodiment, a configuration in which the leg units 14 are pivoted has been described, the leg units 14 may be slid to protrude from the housing 10. The leg units 14 may have any other configuration as long as the leg units 14 support the mobile examination apparatus.

FIG. 1B is the perspective view of the mobile examination apparatus and illustrates a state in which the leg units 14 are pivoted toward the side on which the main arm 18 is set. As described below, because the radiation emitting unit 22, which is heavy, is disposed on a distal end of the main arm 18 when the main arm 18 is unfolded, the mobile examination apparatus may topple over toward the side on which the main arm 18 is set unless supported. The leg units 14 support the mobile examination apparatus by contacting the floor from coupling portions to distal ends or near the distal ends thereof. By rotating the leg units 14 toward the side on which the main arm 18 is set, the mobile examination apparatus is prevented from toppling over toward the radiation emitting unit 22.

Regarding the main arm 18, one end in the longitudinal direction is coupled to the radiation emitting unit 22, and the other end is coupled to the column 16. The main arm 18 includes a first handle 18a that is for the user to hold when transporting the mobile examination apparatus. The first handle 18a may also be referred to as a first grip portion.

The radiation emitting unit 22 is a transmission‑type radiation emitting unit having a function of generating radiation. The radiation emitting unit 22 is implemented using, for example, an X‑ray tube, and emits radiation toward the subject. The transmission‑type radiation emitting unit is provided with shielding members disposed on an electron incidence side and a radiation emitting side of a target to block unnecessary radiation. Since the transmission‑type radiation emitting unit is not required to cover the entire periphery of a radiation generating tube or an outer casing with shielding members made of materials, such as lead, the unit can achieve a smaller size and lighter weight compared to, for example, a rotating anode-type radiation emitting unit.

In the state for transportation as illustrated in FIG. 1A, the main arm 18 is folded relative to the column 16, and the column 16 is slid and moved downward. By positioning the radiation emitting unit 22 near the floor surface, the mobile examination apparatus can achieve a lower center of gravity. With this configuration, the balance of the mobile examination apparatus can be maintained, and an operating force by the user using the first handle 18a to tilt the mobile examination apparatus during transportation is reduced.

During transportation, the main arm 18, the radiation emitting unit 22, and the column 16 are housed in the main‑arm housing unit 10a. In this state, the radiation emitting unit 22 is positioned between the main arm 18 and the column 16, and the main arm 18 serves as a cover for the radiation emitting unit 22. A surface of the main arm 18 opposite to a surface coupling with the radiation emitting unit 22 becomes flush with the side surface of the housing 10 when the main arm 18 is housed, forming part of an external appearance of the mobile examination apparatus. Thus, the external appearance of the mobile examination apparatus has no protrusions, eliminating a risk of snagging during transportation, enabling safe transport.

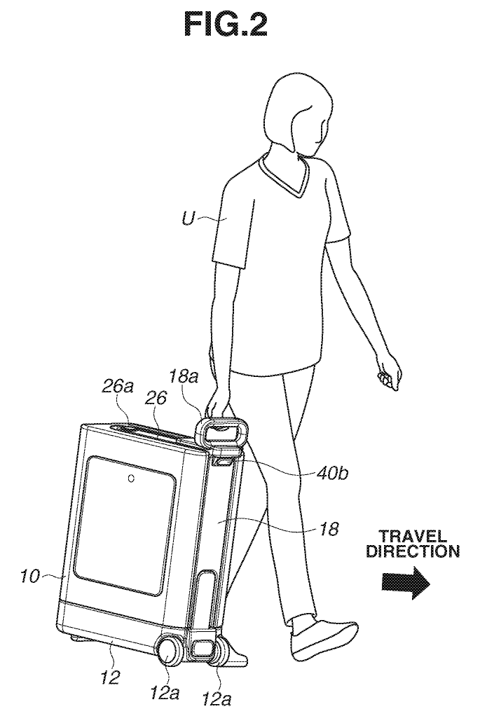

FIG. 2 is a diagram illustrating a state in which the mobile examination apparatus is transported by a user U. As illustrated in FIG. 2, the first handle 18a is disposed at an end (upper end) of the main arm 18 and extends outward beyond an outer contour of the housing 10 so that the user U can easily hold the first handle 18a. The user U holds the first handle 18a and pulls the mobile examination apparatus in a travel direction of the mobile unit 12, causing the plurality of wheels 12a to rotate, so that the mobile examination apparatus can be transported. Because the first handle 18a is disposed substantially parallel to a direction in which the column 16 slides, the user can hold the first handle 18a and move the column 16, to which the main arm 18 is coupled, vertically when installing the mobile examination apparatus.

The main arm 18 supports the radiation emitting unit 22 and has a predetermined length. As illustrated in FIG. 1B, the main arm 18 includes an extension mechanism that allows the main arm 18 to extend and retract in the longitudinal direction (direction B). Thus, the radiation emitting unit 22 can be moved in the longitudinal direction (direction B) of the main arm 18. This configuration enables the main arm 18 to be extended in the longitudinal direction (direction B) and the radiation emitting unit 22 to be positioned away from the housing 10.

Further, the main arm 18 can pivot about the upper end of the column 16. Specifically, as illustrated in FIG. 1B, the column 16 includes a main‑arm hinge portion 20 for pivoting the main arm 18 in a predetermined pivot direction (direction C).

The main‑arm hinge portion 20 couples the main arm 18 to the column 16 and includes a mechanism that allows the main arm 18 to be unfolded and folded relative to the column 16. When the main arm 18 is folded relative to the column 16, the main arm 18 becomes substantially perpendicular to the floor surface and substantially parallel to the column 16.

As described above, the main arm 18 has a folding function via the main‑arm hinge portion 20. In a housed state, the main arm 18 is housed in the housing 10 in a folded state, with the main arm 18 being substantially perpendicular to the floor surface and remaining parallel to the column 16.

In the housed state, the main arm 18 is folded while being retracted in the longitudinal direction (direction B). A retracted length of the main arm 18 is shorter than the height of the housing 10. Therefore, the main arm 18 can be housed in the housing 10. During a radiographic examination, the main arm 18 is substantially parallel to the floor surface and substantially perpendicular to the column 16.

As illustrated in FIG. 1A, in the housed state, the main arm 18 is folded relative to the column 16, and the first handle 18a is disposed at the end (upper end) of the main arm 18. In this state, the first handle 18a is positioned at the upper end of the column 16.

As illustrated in FIG. 1B, during a radiographic examination, the main arm 18 is extended and is substantially parallel to the floor surface. In this state, the first handle 18a is disposed at an end (left end) of the main arm 18.

As described above, the first handle 18a moves in conjunction with movement of the main arm 18, and orientation of the first handle 18a is changed. In other words, the orientation of the first handle 18a is determined based on orientation of the main arm 18.

The sub arm 26 does not have a folding function relative to the column 16. In the housed state, the sub arm 26 is housed in the housing 10 without being folded. During a radiographic examination and in the housed state, the sub arm 26 is substantially parallel to the floor surface. A length of the sub arm 26 is shorter than a width of the housing 10 (the longitudinal direction of the upper surface of the housing 10). Therefore, the sub arm 26 can be housed in the housing 10.

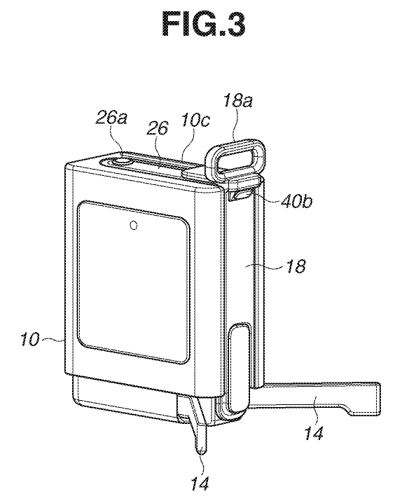

FIG. 3 is a perspective view of the mobile examination apparatus and illustrates a state in which the mobile examination apparatus is placed on the floor surface. As illustrated in FIG. 2, the mobile examination apparatus is transported by the user, and the cover 12b of the mobile unit 12 is removed from the housing 10. The user places the mobile examination apparatus at a desired position and pivots the leg units 14. The mobile examination apparatus is set on the floor surface using the leg units 14 and the bottom surface of the housing 10.

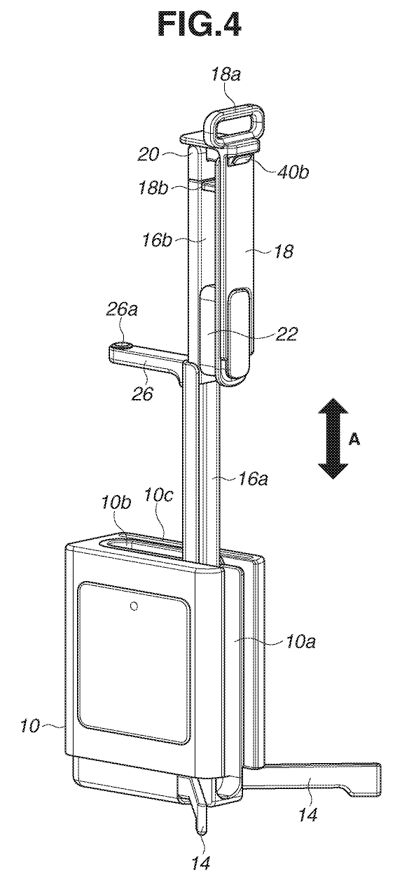

FIG. 4 is a perspective view of the mobile examination apparatus and illustrates a state in which the column 16 has been moved upward. From the housed state illustrated in FIG. 1A, when performing a radiographic examination, the user holds the first handle 18a of the main arm 18 and moves the column 16 upward.

In FIG. 4 illustrating the state in which the column 16 has been moved upward, the main arm 18 is folded relative to the column 16, and the first handle 18a is disposed at the end (upper end) of the main arm 18. In this state, the first handle 18a is at the upper end of the column 16.

Then, the user unfolds the main arm 18. The main arm 18 becomes substantially parallel to the floor surface. The main arm 18 has approximately the same length as the extendable portion 16b. Further, by extending the main arm 18, the radiation emitting unit 22 protrudes outward so that the radiation emitting unit 22 can be positioned above an examination site of the subject, as illustrated in FIG. 1B.

The main arm 18 has an extension and retraction mechanism in the longitudinal direction (direction B). Further, the main arm 18 includes a second handle 18b which is held by the user to extend or retract the main arm 18. The second handle 18b may also be referred to as a second grip portion.

During a radiographic examination, the second handle 18b (second grip portion) is disposed on the same surface as an emitting surface of the radiation emitting unit 22 so that the user easily holds the second handle 18b when the main arm 18 is unfolded. The second handle 18b is configured in such a manner that the second handle 18b is disposed on a lower surface of the main arm 18 when the main arm 18 is unfolded to be substantially parallel to the floor surface during a radiographic examination.

By holding the second handle 18b disposed on the lower surface of the main arm 18 and extending and retracting the main arm 18, the user can easily adjust alignment between the subject lying on a bed and the radiation emitting unit 22 coupled to the main arm 18.

According to the present embodiment, the main arm 18 (first arm) includes the first handle 18a (first grip portion) at the end of the main arm 18 and the second handle 18b (second grip portion) on the same surface as the emitting surface of the radiation emitting unit 22.

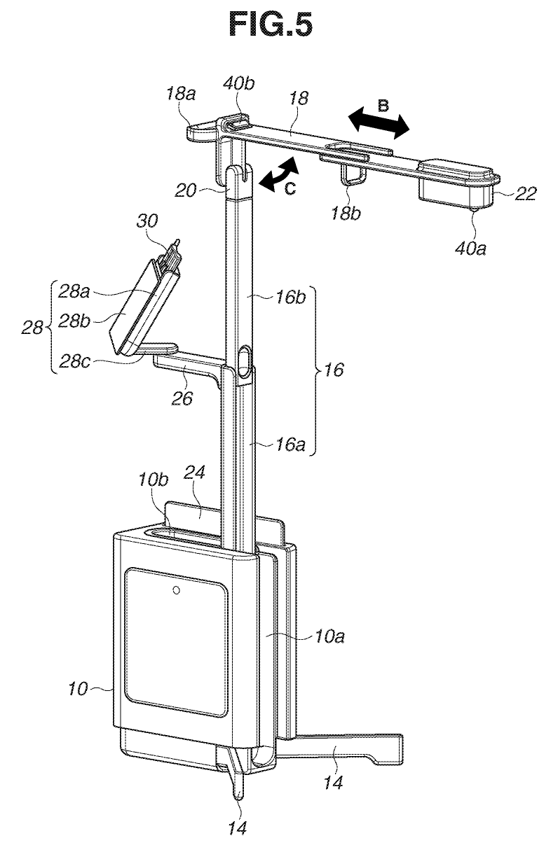

FIG. 5 is a perspective view of the mobile examination apparatus and illustrates a state in which the radiation detector 24 has been taken out from the housing 10. The radiation detector 24 is capable of detecting radiation emitted from the radiation emitting unit 22. The radiation detector 24 is a flat panel detector (FPD) in a plate shape. The housing 10 includes the radiation detector housing unit 10c in the upper surface. The radiation detector housing unit 10c is a space for housing the radiation detector 24 and is formed so that the radiation detector 24 in a plate shape fits along the side surface of the housing 10. The user can remove the radiation detector 24 by gripping one end of the radiation detector 24 from the upper surface of the housing 10.

The sub arm 26 is coupled to the column 16. Specifically, the sub arm 26 is coupled to the movable portion 16a that moves in the vertical direction of the column 16. By moving the movable portion 16a in the vertical direction, the sub arm 26 can be moved in the vertical direction. The sub arm 26 is housed in the sub‑arm housing unit 10b of the housing 10 and becomes flush with the upper surface of the housing 10 when being housed. The sub arm 26 forms part of the upper surface of the mobile examination apparatus (housing 10). In other words, the sub arm 26 forms part of the exterior of the mobile examination apparatus (housing 10). As illustrated in FIG. 4, the sub arm 26 includes an engagement portion 26a for attaching the panel unit 28 in a vicinity of a distal end. The panel unit 28 is disposed on the sub arm 26 via the engagement portion 26a. The panel unit 28 includes a tablet computer including a display unit and an input unit.

The sub arm 26 can be moved by the movable portion 16a of the column 16 independently of the vertical movement of the column 16, and thus, the user can adjust a height of the panel unit 28. The panel unit 28 includes an ultrasound probe housing 28a, a touch panel 28b, and a stand 28c as will be described below. The panel unit 28 can hold the ultrasound probe 30 thereon.

While, in the present embodiment, the configuration in which the sub arm 26 is coupled to the column 16 has been described, the sub arm 26 may be coupled to a component other than the column 16. For example, another column may be provided below the sub arm 26 and coupled to the housing 10 so that the sub arm 26 can be moved vertically. In this case, the sub arm 26 alone can be moved upward without moving the column 16 upward.

According to the present embodiment, the mobile examination apparatus includes the sub arm 26 (second arm) on which the panel unit 28 is disposed, and the sub arm 26 is disposed on the column 16 or the housing 10 to be movable in the vertical direction. The housing 10 includes the sub‑arm housing unit 10b (third housing unit) for housing at least a portion of the sub arm 26. When the sub arm 26 is housed in the housing 10, at least a portion of the sub arm 26 forms part of the housing 10.

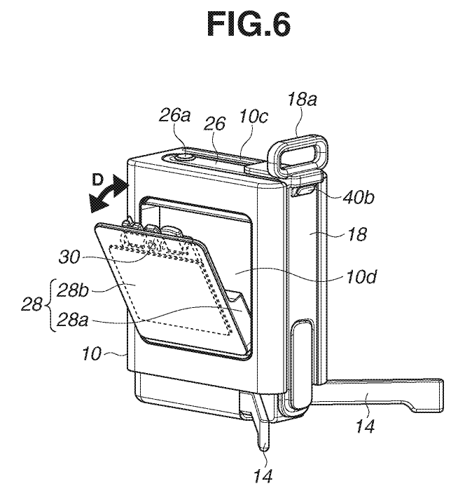

FIG. 6 is a perspective view of the mobile examination apparatus and illustrates a state in which a side surface of the housing 10 is opened. The housing 10 includes a panel‑unit housing unit 10d in the side surface. The panel‑unit housing unit 10d may also be referred to as a second housing unit.

The panel‑unit housing unit 10d (second housing unit) is a space for housing the panel unit 28 and allows the user to take the panel unit 28 out when the side surface of the housing 10 is opened in a direction D. In other words, the panel‑unit housing unit 10d (second housing unit) allows the panel unit 28 to be housed by opening and closing the side surface of the housing 10. The panel unit 28 can pivot in the direction D about a lower end thereof.

The panel unit 28 includes the ultrasound probe housing 28a for housing the ultrasound probe 30, the touch panel 28b, and the stand 28c. The panel unit 28 includes an engagement portion on the stand 28c which is on a side (rear side) opposite a side with the touch panel 28b. The panel unit 28 is attached to the engagement portion 26a of the sub arm 26 illustrated in FIG. 4. One end of the sub arm 26 serves as a portion for coupling to the column 16, and the other end of the sub arm 26 serves as the engagement portion 26a. The panel unit 28 is disposed at a position separated from the column 16 by a predetermined distance (corresponding to the length of the sub arm 26).

According to the present embodiment, the housing 10 includes the panel‑unit housing unit 10d (second housing unit) for housing the panel unit 28 on which the ultrasound probe 30 for transmitting and receiving ultrasound waves is placed. The panel‑unit housing unit 10d (second housing unit) enables the panel unit 28 to be housed by opening and closing the side surface of the housing 10.

FIG. 1B illustrates a state in which the panel unit 28 is tilted. An angle of the panel unit 28 can be changed by swinging the panel unit 28 relative to the sub arm 26 according to a user's application. An example of the swinging operation for changing the angle of the panel unit 28 is a tilting operation.

Further, a rotation axis of the engagement portion 26a is parallel to the longitudinal direction of the column 16. Thus, the panel unit 28 is rotatable about an axis parallel to the longitudinal direction of the column 16. The panel unit 28 is rotatable at the end of the sub arm 26. A rotation mechanism allows the panel unit 28 to rotate within a predetermined rotation range (for example, 180 degrees (°)). The rotation mechanism of the panel unit 28 has a function of maintaining the rotational position of the panel unit 28 at any desired position.

The ultrasound probe housing 28a is a placing unit for placing the ultrasound probe 30 to be used in an ultrasound examination. The touch panel 28b displays generated radiographic images, ultrasound images, and the like, and displays a graphical user interface (GUI) via which the user inputs various instructions. The touch panel 28b can also receive instructions input by the user. The user adjusts a height of the sub arm 26, on which the panel unit 28 has been attached, in accordance with the user's posture during an examination so that the user can easily remove the ultrasound probe 30 and operate the touch panel 28b. Further, the user can adjust the touch panel 28b to a position where the user can easily view the touch panel 28b during an examination by rotating the panel unit 28 horizontally about the engagement portion 26a.

While, in the present embodiment, the configuration in which the ultrasound probe housing 28a and the touch panel 28b are integrated in the panel unit 28 has been described, the touch panel 28b may be configured to be detachable.

The radiation emitting unit 22 includes a first camera 40a for capturing an image of the subject. The main arm 18 includes a second camera 40b for capturing an image of the subject. The second camera 40b is disposed at one end of the main arm 18, in a vicinity of the main‑arm hinge portion 20, on a surface forming an exterior of the main arm 18.

The mobile examination apparatus according to the present disclosure includes the housing 10, the column 16 connected to the housing 10 and movable in the vertical direction, the first arm (main arm 18) that is coupled to the column 16, supports the radiation emitting unit 22 for emitting radiation, and is extendable and retractable in the longitudinal direction, and the mobile unit 12 that supports the housing 10 and is movable on the floor surface. The first arm (main arm 18) is foldable relative to the column 16, and the housing 10 includes the first housing unit (main‑arm housing unit 10a) for housing at least a portion of the radiation emitting unit 22, the first arm (main arm 18), and the column 16.

When the first arm (main arm 18) is housed in the housing 10, at least a portion of the first arm (main arm 18) forms part of the housing. Therefore, a mobile examination apparatus that can be easily installed can be provided.

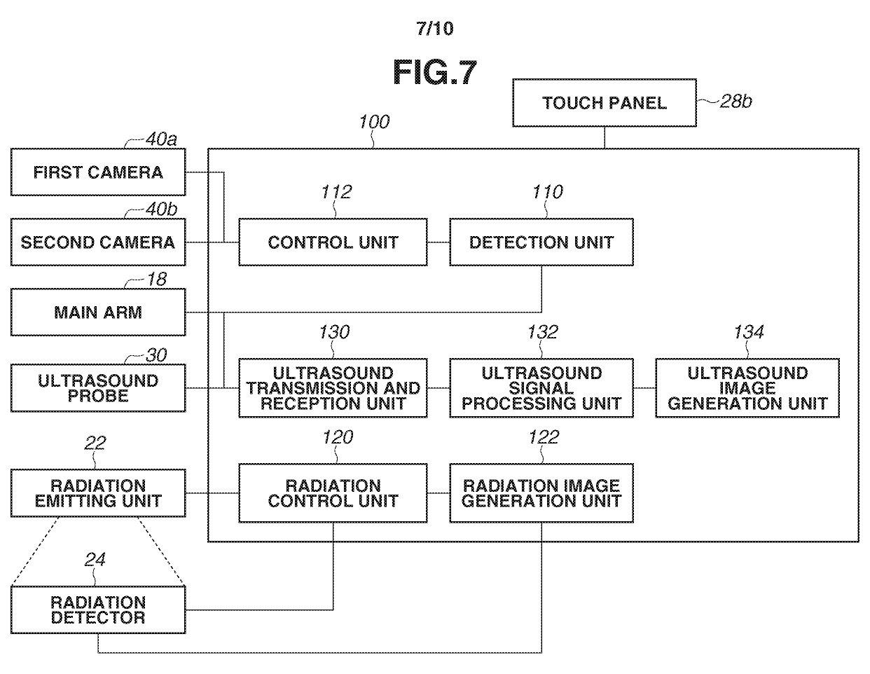

An internal configuration of the mobile examination apparatus will be described with reference to FIG. 7. FIG. 7 is a diagram illustrating a configuration of an apparatus main body 100 of the mobile examination apparatus. The apparatus main body 100 is provided inside the housing 10.

The apparatus main body 100 includes a detection unit 110 for detecting states of the main arm 18 and the ultrasound probe 30, a control unit 112 for controlling various components, a radiation control unit 120 for controlling the radiation emitting unit 22 and the radiation detector 24, a radiation image generation unit 122 for generating radiographic image data using image signals transmitted from the radiation detector 24, an ultrasound transmission and reception unit 130 for causing the ultrasound probe 30 to transmit and receive ultrasound, an ultrasound signal processing unit 132 for performing various types of signal processing using ultrasound signals based on reflected wave signals received by the ultrasound transmission and reception unit 130, and an ultrasound image generation unit 134 for generating an ultrasound image using signal processing data processed by the ultrasound signal processing unit 132.

The detection unit 110 detects that the main arm 18 has been pivoted and unfolded or extended as illustrated in FIG. 1B and a preparation for a radiographic examination has started. Examples of the detection method include a mechanism using a sensor in the main‑arm hinge portion 20 or the main arm 18 to detect that the main arm 18 has been unfolded from the column 16 or to detect that the main arm 18 has been extended. The detection unit 110 also detects that the ultrasound probe 30 has been removed from the panel unit 28 and a preparation for an ultrasound examination has started. Examples of the detection method include a mechanism using a sensor in the ultrasound probe housing 28a of the panel unit 28 to detect that the ultrasound probe 30 has been removed.

When the detection unit 110 detects that a preparation for a radiographic examination or an ultrasound examination has started, the control unit 112 controls image capturing by the first camera 40a and the second camera 40b and display on the touch panel 28b. Specifically, the control unit 112 starts capturing images by the first camera

40a and the second camera 40b and causes the captured images to be displayed on the touch panel 28b. The user performs a pre‑examination preparation, such as alignment between the radiation emitting unit 22 and the radiation detector 24 for a radiographic examination, while viewing the images captured by the first camera 40a from above the subject. Since the first camera 40a captures the subject from above, an examination site of the subject is easily recognized, and the alignment can be easily performed. The control unit 112 can acquire various types of information about the subject using a recognition technology of the camera. For example, by detecting pulse waves of the subject’s face, hand, or the like, vital information, such as a pulse rate or a blood pressure, can be obtained. Furthermore, when the detection unit 110 detects that a preparation for a radiographic examination or an ultrasound examination has started, the control unit 112 activates an application to be used for the radiographic examination or the ultrasound examination and displays the application on the touch panel 28b.

The radiation control unit 120 has a function of controlling the radiation emitting unit 22. The radiation control unit 120 sets imaging conditions (tube voltage, tube current, irradiation time) for radiation to be emitted from the radiation emitting unit 22. The radiation control unit 120 can also control the timing at which the radiation emitting unit 22 emits radiation and the timing at which the radiation detector 24 captures an image. The radiation control unit 120 can cause the radiation emitting unit 22 to emit radiation multiple times, and the radiation detector 24 can capture images multiple times in accordance with the emissions from the radiation emitting unit 22.

As described above, the radiation emitting unit 22 can emit radiation to a desired area. The radiation detector 24 can detect radiation emitted from the radiation emitting unit 22, as described above. The radiation detector 24 includes an array of pixels (arranged in a two-dimensional plane region) each of which includes an image sensor element that outputs a radiation signal corresponding to radiation (incident light) emitted from the radiation emitting unit 22. A photoelectric conversion element of each of the pixels converts light converted by a phosphor into a radiation signal, which is an electrical signal (hereinbelow also referred to as an image signal), and a capacitor of each of the pixels accumulates charges of radiation signals (image signals). The radiation detector 24 reads out the image signals accumulated in the capacitor of each of the pixels and transmits the image signals to the radiation image generation unit 122.

The radiation image generation unit 122 performs image processing, such as tone processing and noise reduction processing, on the image signals transmitted from the radiation detector 24. Then, the radiation image generation unit 122 transmits radiation image data generated after the image processing to the touch panel 28b.

The ultrasound probe 30 is connected to the apparatus main body 100 via wireless communication. The ultrasound probe 30 includes a plurality of transducers and can generate ultrasound waves by driving the plurality of transducers. The ultrasound probe 30 receives reflected ultrasound waves from the subject and converts them into electrical signals. The converted electrical signals are transmitted to the apparatus main body 100.

The ultrasound probe 30 includes an acoustic matching layer that is provided on a front side (subject side) of the plurality of transducers to match the acoustic impedance between the plurality of transducers and the subject, and a backing material that is provided on a rear side of the plurality of transducers to prevent propagation of ultrasound waves toward the rear side. While, in the present embodiment, wireless communication is performed between the ultrasound probe 30 and the apparatus main body 100, a connector receiving unit capable of being connected to a connector of the ultrasound probe 30 may be provided in the housing 10 to enable wired communication.

The ultrasound transmission and reception unit 130 controls transmission and reception of ultrasound waves that are performed by the ultrasound probe 30. The ultrasound transmission and reception unit 130 includes a transmission unit, a transmission delay circuit, and the like, and supplies drive signals to the ultrasound probe 30. The transmission unit repetitively generates rate pulses at a predetermined pulse repetition frequency (PRF). The transmission delay circuit applies a delay time to the rate pulses generated by the transmission unit in order to cause ultrasound waves emitted from the ultrasound probe 30 to be focused and determine transmission directivity. By varying the delay time to be applied to the rate pulses, the transmission delay circuit can control a transmission direction of ultrasound waves transmitted from the transducers.

The ultrasound transmission and reception unit 130 includes an amplifier, an analog/digital (A/D) conversion unit, a reception delay circuit, and an addition unit. Various types of processing are performed on the reflected wave signals received by the ultrasound probe 30 to generate ultrasound signals. The amplifier amplifies the reflected wave signals for each channel and performs gain correction processing. The A/D conversion unit converts the gain-corrected reflected wave signals into digital data.

The reception delay circuit applies a delay time to the digital data to determine reception directivity. The addition unit performs summation processing on the reflected wave signals to which delay times have been applied by the reception delay circuit. Through the summation processing by the addition unit, reflection components from a direction corresponding to the reception directivity of the reflected wave signals are emphasized.

When performing two-dimensional scanning of the subject, the ultrasound transmission and reception unit 130 causes the ultrasound probe 30 to transmit two-dimensional ultrasound waves. Then, the ultrasound transmission and reception unit 130 generates two-dimensional ultrasound signals from two-dimensional reflected wave signals received by the ultrasound probe 30. When performing three-dimensional scanning of the subject, the ultrasound transmission and reception unit 130 causes the ultrasound probe 30 to transmit three-dimensional ultrasound waves. Then, the ultrasound transmission and reception unit 130 generates three-dimensional ultrasound signals from the three-dimensional reflected wave signals received by the ultrasound probe 30.

The ultrasound signal processing unit 132 performs various types of signal processing on the ultrasound signals output from the ultrasound transmission and reception unit 130. Specifically, the ultrasound signal processing unit 132 performs signal processing, such as envelope detection processing or logarithmic compression, on the ultrasound signals. The ultrasound signal processing unit 132 visualizes amplitude information on the ultrasound signals and generates signal processing data (raster data). Band-pass filter processing is applied to the ultrasound signals output from the ultrasound transmission and reception unit 130, and then envelopes of the output signals are detected. The envelope detected data is then subjected to compression processing by logarithmic conversion. The ultrasound signal processing unit 132 outputs the signal processing data obtained by the signal processing to the ultrasound image generation unit 134.

The ultrasound image generation unit 134 generates an ultrasound image using the signal processing data processed by the ultrasound signal processing unit 132. The ultrasound image generation unit 134 includes a digital scan converter, which converts the signal processing data into data represented in orthogonal coordinates. In the present embodiment, the ultrasound image generation unit 134 converts the signal processing data (raster data) into a Cartesian coordinate system (X, Y) of display image data. Then, the ultrasound image generation unit 134 generates an ultrasound image (B- mode image data) in which signal intensity is displayed as brightness. In this way, the ultrasound image generation unit 134 performs generation of the ultrasound image. As an ultrasound image generation algorithm, not only phase alignment summation processing but also any algorithm may be employed to perform image reconstruction.

### Radiographic Examination

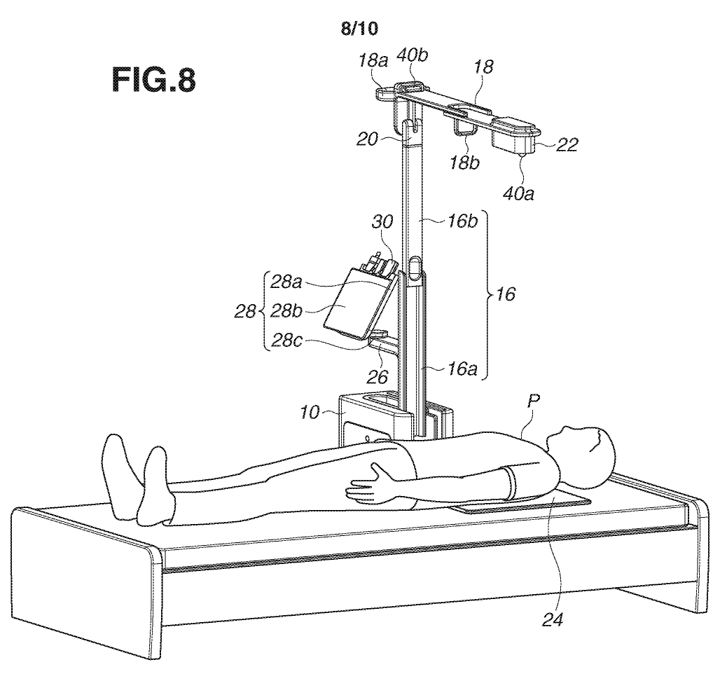

FIG. 8 is a diagram illustrating an example of a radiographic examination using the mobile examination apparatus according to the present embodiment. A subject P is lying on a bed, and the mobile examination apparatus is installed beside the subject P. The user is not illustrated in FIG. 8 because the user stays away from the bed to avoid exposure from the radiation emitting unit 22. The column 16 has been moved upward, the main arm 18 has been unfolded to be substantially perpendicular to the column 16 and extended so that the radiation emitting unit 22 protrudes. In this way, when performing a radiographic examination, the user can easily position the radiation emitting unit 22 by extending the column 16 and unfolding the main arm 18 without attaching the radiation emitting unit 22 to another holder provided above the subject P.

The radiation emitting unit 22 is positioned above the subject P, and the radiation detector 24 is positioned below the subject P. In response to the main arm 18 being unfolded, the control unit 112 displays, on the touch panel 28b, images captured by the first camera 40a disposed in the vicinity of the radiation emitting unit 22 and the application to be used for a radiographic examination. The user adjusts the radiation emitting unit 22 so that the radiation emitting unit 22 is positioned above an examination site of the subject P, and adjusts the radiation detector 24 so that radiation detector 24 is positioned below the examination site of the subject P, while viewing the touch panel 28b. In this way, the user can view the image captured from above the subject P and easily perform alignment between the radiation emitting unit 22 and the radiation detector 24. Further, since the first camera 40a performs image capturing of the subject P from above, vital information, such as pulse waves acquired from the face of the subject P, can be obtained. The user can check the vital information without using another device.

### Ultrasound Examination

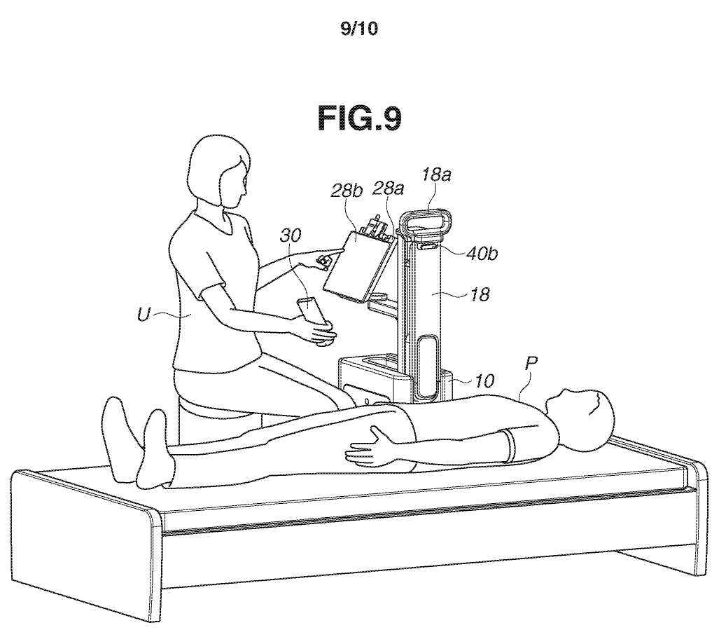

FIG. 9 is a diagram illustrating an example during an ultrasound examination using the mobile examination apparatus according to the present embodiment. The subject P is lying on the bed, and the mobile examination apparatus is installed beside the subject P. The user U is seated on a chair next to the bed and performs an ultrasound examination on the subject P using the ultrasound probe 30. The column 16 has been moved upward so that the second camera 40b can easily capture images of the subject P, and the sub arm 26 is positioned at a level that allows the user U to easily operate the touch panel 28b. In response to the ultrasound probe 30 being removed, the control unit 112 displays, on the touch panel 28b, images captured by the second camera 40b disposed at the upper end of the main arm 18 and the application to be used for an ultrasound examination. In this way, during an ultrasound examination, the user U can perform the examination in a natural posture while viewing captured images of the subject P and ultrasound images displayed on the touch panel 28b.

Since the second camera 40b captures images from above the subject P, vital information can be acquired from, for example, a pulse wave of the subject P’s face. The user U can check the vital information without using another device.

When a radiographic examination or an ultrasound examination is finished, the user U houses the radiation emitting unit 22, the radiation detector 24, the ultrasound probe 30, and the panel unit 28 in the housing 10. After moving the housing 10 from the subject P’s home to an outdoor location, the mobile unit 12 is attached to the bottom surface of the housing 10, and the mobile examination apparatus is transported as illustrated in FIG. 2.

As described above, the mobile examination apparatus according to the present disclosure has excellent housing capability for the components to be used for an examination, which improves handling during transportation.

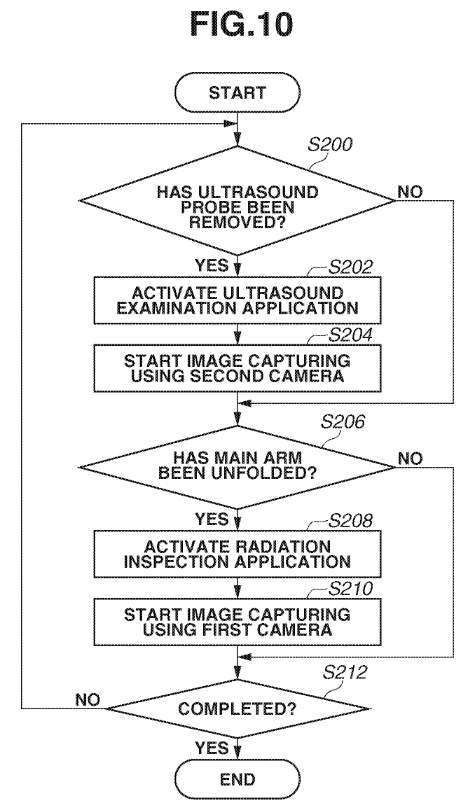

FIG. 10 is a flowchart illustrating an operation during installation of the mobile examination apparatus according to the present embodiment. FIG. 10 illustrates the operation that is performed when the user installs the mobile examination apparatus beside a subject and performs a radiographic examination or an ultrasound examination.

In step S200, the detection unit 110 determines whether the ultrasound probe 30 has been removed from the panel unit 28. In a case where the detection unit 110 determines that the ultrasound probe 30 has not been removed (NO in step S200), the processing proceeds to step S206. In a case where the detection unit 110 determines that the ultrasound probe 30 has been removed (YES in step S200), the processing proceeds to step S202.

In step S202, the control unit 112 activates the application to be used for the ultrasound examination and causes the application to be displayed on the touch panel 28b.

That is, when the detection unit 110 determines that the ultrasound probe 30 has been removed from the panel unit 28, the application to be used for the ultrasound examination is activated and the application is displayed on the touch panel 28b.

In step S204, the control unit 112 starts image capturing using the second camera 40b and causes the captured images and vital information to be displayed on the touch panel 28b. In this processing, on the touch panel 28b, the application for an ultrasound examination and captured images and vital information acquired by the second camera 40b may be displayed side by side horizontally or vertically, or superimposed to allow the user to select.

By activating the application and starting image capturing using the second camera 40b as the ultrasound probe 30 is removed from the panel unit 28, the user’s workload can be reduced, and the examination can be started without keeping the subject waiting.

In step S206, the detection unit 110 determines whether the main arm 18 has been unfolded. In a case where the detection unit 110 determines that the main arm 18 has not been unfolded (NO in step S206), the processing proceeds to step S212. In a case where the detection unit 110 determines that the main arm 18 has been unfolded (YES in step S206), the processing proceeds to step S208. When the processing proceeds to step S208, the flowchart ends if the application for an ultrasound examination and the second camera 40b are already active.

In step S208, the control unit 112 activates an application to be used for a radiographic examination and causes the application to be displayed on the touch panel 28b. That is, in a case where the detection unit 110 determines that the main arm 18 has been unfolded, the application for the radiographic examination is activated and displayed on the touch panel 28b.

In step S210, the control unit 112 starts image capturing using the first camera 40a and causes the captured images to be displayed on the touch panel 28b. In this processing, on the touch panel 28b, the application for a radiographic examination and the images captured by the first camera 40a may be displayed side by side horizontally or vertically, or superimposed to allow the user to select.

By activating the application and starting image capturing using the first camera 40a as the main arm 18 is unfolded, the user’s workload can be reduced, and the alignment between the radiation emitting unit 22 and the radiation detector 24 and the start of the radiographic examination can be performed without keeping the subject waiting.

In step S212, determination of whether the user’s operation has been completed is performed. For example, an end button is displayed on the touch panel 28b, and completion of the operation is determined in a case where the user touches the end button. In a case where it is determined that the operation has been completed (YES in step S212), the application for an ultrasound examination and the second camera 40b, which are active, or the application for a radiographic examination and the first camera 40a, which are active, are terminated. In a case where it is determined that the operation has not been completed (NO in step S212), the processing returns to step S200.

According to the present embodiment, the radiation emitting unit 22 includes the first camera 40a for capturing images of the subject. The mobile examination apparatus further includes the detection unit 110 for detecting that the first arm (main arm 18) has been rotated or extended, and the control unit 112 for starting, in response to detection by the detection unit 110 that the first arm has been rotated or extended, capturing images using the first camera 40a or activating the application to be used for a radiographic examination.

The first arm (main arm 18) includes the second camera 40b for capturing images of the subject. The mobile examination apparatus further includes the detection unit 110 for detecting that the ultrasound probe 30 has been removed from the panel unit 28, and the control unit 112 for starting, in response to detection by the detection unit 110 that the ultrasound probe 30 has been removed from the panel unit 28, capturing images using the second camera 40b or activating the application to be used for an ultrasound examination.

A computer program for implementing the functions of the above-described embodiment may be supplied to a computer via a network or a memory (not illustrated), and a processor (not illustrated) may execute the computer program. The above-described information processing method is implemented by causing the computer to execute the computer program. That is, the computer program is a program for causing a computer to realize the functions of the information processing apparatus. The memory stores the computer program.

The present disclosure is not limited to the above-described embodiment and various modifications can be made without departing from the spirit and scope of the disclosure. Therefore, the following claims are appended to publicly disclose the scope of the present invention.

According to the present disclosure, a mobile examination apparatus that is easy to install can be provided.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a 'non-transitory computer-readable storage medium') to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)TM), a flash memory device, a memory card, and the like.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

1. A mobile examination apparatus comprising:
a housing;
a column coupled to the housing and configured to extend and retract in a vertical direction;
a first arm coupled to the column and configured to extend and retract in a longitudinal direction;
a radiation emitting unit supported by the first arm and configured to emit radiation; and
a mobile unit configured to support the housing and move on a floor surface,
wherein the first arm is folded relative to the column while supporting the radiation emitting unit, and the housing houses the radiation emitting unit, the first arm, and the column therein.

2. The mobile examination apparatus according to claim 1, wherein the housing includes a first housing unit for housing at least a portion of the radiation emitting unit, the first arm, and the column.

3. The mobile examination apparatus according to claim 1, wherein when the first arm is housed in the housing, at least a portion of the first arm forms part of the housing.

4. The mobile examination apparatus according to claim 1, wherein the first arm includes a first grip portion at an end thereof and a second grip portion on a same surface as an emitting surface of the radiation emitting unit.

5. The mobile examination apparatus according to claim 1, wherein the mobile unit is detachable from the housing.

6. The mobile examination apparatus according to claim 1, wherein the housing includes a second housing unit for housing a panel unit on which an ultrasound probe for transmitting and receiving ultrasound waves is held.

7. The mobile examination apparatus according to claim 6, wherein the second housing unit is configured to house the panel unit by opening and closing a side surface of the housing.

8. The mobile examination apparatus according to claim 6, wherein the panel unit includes a tablet computer having a display unit and an input unit.

9. The mobile examination apparatus according to claim 6, further comprising a second arm configured to support the panel unit,
wherein the second arm is disposed on the column or the housing to be movable in the vertical direction.

10. The mobile examination apparatus according to claim 9, wherein the housing includes a third housing unit for housing at least a portion of the second arm.

11. The mobile examination apparatus according to claim 10, wherein when the second arm is housed in the housing, at least a portion of the second arm forms part of the housing.

12. The mobile examination apparatus according to claim 9, wherein the first arm and the second arm are disposed in directions different from each other relative to the column.

13. The mobile examination apparatus according to claim 9, wherein the second arm is not folded relative to the column.

14. The mobile examination apparatus according to claim 11, wherein the housing includes the first housing unit in a side surface of the housing and the third housing unit in an upper surface of the housing.

15. The mobile examination apparatus according to claim 1, wherein the housing includes a fourth housing unit for housing a radiation detector for detecting radiation emitted from the radiation emitting unit.

16. The mobile examination apparatus according to claim 1, wherein the radiation emitting unit includes a first camera for capturing an image of a subject.

17. The mobile examination apparatus according to claim 16, further comprising:
a detection unit configured to detect that the first arm has been rotated or extended; and
a control unit configured to start, in a case where the detection unit detects that the first arm has been rotated or extended, image capturing using the first camera.

18. The mobile examination apparatus according to claim 1, further comprising:
a detection unit configured to detect that the first arm has been rotated or extended; and
a control unit configured to activate, in a case where the detection unit detects that the first arm has been rotated or extended, an application to be used for a radiographic examination.

19. The mobile examination apparatus according to claim 6, wherein the first arm includes a second camera for capturing an image of a subject.

20. The mobile examination apparatus according to claim 19, further comprising:
a detection unit configured to detect that the ultrasound probe has been removed from the panel unit; and
a control unit configured to start, in a case where the detection unit detects that the ultrasound probe has been removed from the panel unit, capturing of an image using the second camera.

21. The mobile examination apparatus according to claim 6, further comprising:
a detection unit configured to detect that the ultrasound probe has been removed from the panel unit; and
a control unit configured to activate, in a case where the detection unit detects that the ultrasound probe has been removed from the panel unit, an application to be used for an ultrasound examination.
